(19) United States
(12) Patent Application Publication
(10) Pub. No.: US 2021/0294946 A1
(43) Pub. Date: Sep. 23, 2021

(54) SELECTING AND APPLYING DIGITAL TWIN MODELS

(71) Applicant: KONINKLIJKE PHILIPS N.V., EINDHOVEN (NL)

(72) Inventors: CORNELIS PETRUS HENDRIKS, EINDHOVEN (NL); MURTAZA BULUT, EINDHOVEN (NL); LIEKE GERTRUDA ELISABETH COX, EINDHOVEN (NL); VALENTINA LAVEZZO, HEEZE (NL)

(21) Appl. No.: 17/145,732

(22) Filed: Jan. 11, 2021

Related U.S. Application Data
(60) Provisional application No. 62/991,614, filed on Mar. 19, 2020.

(51) Int. Cl. G06F 30/27

(52) U.S. Cl. CPC G06F 30/27

(57) ABSTRACT

Embodiments are described herein for selecting and applying models of digital twins for various purposes. In various embodiments, one or more user needs of a user seeking to utilize a digital twin may be identified. One or more situational needs may be identified of a subject simulated by the digital twin. The one or more situational needs may be identified based on data obtained from the subject. Based on one or more of the user needs and one or more of the situational needs of the subject, one or more models of the digital twin may be selected and applied to generate digital twin output that simulates one or more aspects of the subject.

### TECHNICAL FIELD

Various embodiments described herein are directed generally to data analysis and application thereof. More particularly, but not exclusively, various methods and apparatus disclosed herein relate to selecting and applying models of digital twins for various purposes.

### BACKGROUND

A digital twin is a collection of mathematical models, functions, parameters, etc., that can be used individually and/or collectively to simulate aspects of a subject of the digital twin. For example, inputs may be applied to a digital twin to generate digital twin output that simulates the underlying subject's behavior, reaction, performance, etc. Digital twins may be built for organic subjects (e.g., people, animals, specific organs or physiological systems) and/or non-organic subjects (e.g., machines, vehicles, aircraft, etc.). One or more aspects of a digital twin may be tailored towards the subject it simulates. For example, parameter(s) of a machine learning model of the digital twin, or aspects of a knowledge graph of the digital twin, may be determined (e.g., set, trained, etc.) at least in part using training data specific to that subject. Consequently, one subject's digital twin may not necessarily be usable to simulate aspect(s) of another subject.

With increasing amounts of available data, mathematical models, and potential applications of those models, it is becoming more difficult to determine which functions and/or models of a digital twin to apply. For example, digital twin models of the human body are often used in clinical practice. Development of these models is driven in large part by clinical needs in diagnosis and treatment. Clinicians and commercial software providers often collaborate to select and implement models in specific clinical applications. Non-limiting examples of models include deep learning algorithms to perform computer-aided detection in radiology, biophysical models for surgery planning, or risk stratification algorithms for readmission prevention. As more models become available, e.g., in artificial intelligence (“AI”) marketplaces, it will become more difficult for clinicians to keep track of all the new model options. Consequently, the selection of an appropriate model by a clinician for implementation and usage in a clinical application has become more difficult.

Patients simulated by digital twins are also gaining increasing access to these digital twin models and/or the output they produce, e.g., as part of healthcare applications that are installed on computing devices (e.g., smart phones, smart watches) of the patients. However, patients who are not medical experts may be even less suited to keep track of and/or select mathematical models that are available with their digital twins than clinicians. Moreover, some patients may not wish to take advantage of every available function/model of their digital twin. For example, some patients may not want to be informed of diagnoses, risks, etc., and/or may wish to maintain control over their private medical data.

### SUMMARY

As noted previously, new models become available all the time, and the ever-increasing volume and variety of available models is enough to overwhelm even experts such as experienced clinicians, much less non-experts such as patients. Accordingly, the present disclosure is directed to methods and apparatus for selecting and applying models of digital twins for various purposes. For example, in various embodiments, a digital twin of a subject may include and/or have access to multiple different models, such as machine learning models. These models may be applicable to make various inferences about the subject and/or the aspect of the subject that is modeled by the digital twin, e.g., based on various simulation parameters being applied as input(s) to the digital twin.

Models may be selected based on a variety of different signals. In the healthcare context, an electronic medical record (“EMR”) and/or electronic health record (“EHR”) may provide various pieces of information (signals) about a subject, such as vital sign measurements, diagnoses, demographics, behavioral information, lab results, nutritional habits, physical activity information, prescriptions, and so forth. One or more sensors may also provide various physiological information about a subject. These sensors may include, but are not limited to, heart rate sensors, blood oxygen sensors, glucose sensors, thermometers, sweat sensors, and so forth.

Other types of signals may include what are referred to as “needs.” Needs may be user-defined (the user being, for instance, a clinician or a subject of the clinician in the medical context) or defined by the current situation/context. For example, a user may operate an application or “app” that allows the user to input what will be referred to herein as “user needs.” These user needs may include, in the healthcare context, types of information that the user wants to receive and doesn't want to receive, and/or the types of information that the user wants to be generated or not generated in the background (e.g., based on privacy concerns).

“Situational needs,” by comparison, are identified based on data obtained from the subject, e.g., via EMRs, EHRs, sensors, dialog with a clinician, etc. As the name implies, situational needs may change over time based on circumstances and/or condition of the subject (user needs may also change over time in some cases, e.g., in response to changing situational needs). In some embodiments, situational and/or user needs may change as part of an overall feedback loop that also includes digital twin output. For example, situational needs may be updated based on output of one or more digital twins. Similarly, a user may be prompted to reconsider his or her user needs based on output of one or more digital twin models. For example, if output of one digital twin model suggests the user (e.g., a patient) may be developing a health condition, the user may be prompted to reconsider one or more user needs that is currently blocking application of another digital twin model that is pertinent to that health condition.

In some embodiments, multiple digital twins may be networked together, such that a “user” of one digital twin may, in fact, be another digital twin. In some such embodiments, at least one human being will still be in control of one or more of those networked digital twins, e.g., to maintain control over privacy and/or ethical considerations.

While examples described herein generally relate to healthcare, this is not meant to be limiting. Techniques described herein may be applicable to select digital twin models in a variety of different contexts and/or domains. For example, a digital twin may be generated for a complex non-organic system such as a vehicle. Different models of/available to the vehicle digital twin may be applicable to make various inferences and/or predict various behaviors and/or outcomes of the vehicle, such as oil life, wear and tear, maintenance prediction, failure prediction, useful life of the vehicle, useful life of vehicle parts, etc. Numerous other applications of techniques described herein are contemplated in myriad different domains.

Various parameters can be applied and/or input to a digital twin to cause the digital twin to generate output that simulates behavior of the digital twin's real-world counterpart. Parameters may simulate real-world parameters that are applicable to a subject and/or to subcomponents (e.g., anatomical structure(s)) within the subject, with the goal of simulating a physical and/or behavioral response of the subject. In the healthcare context, parameters may include, for instance, simulated administration of medicine (e.g., type, dosage, conditions under which it is administered), simulated application of therapy (e.g., physical therapy, implants and/or prosthetics, organ transplants, etc.), simulated activity (e.g., exercise and/or stretching prescribed by a clinician), simulated nutritional intake (e.g., high protein, low carb diet, etc.), daily activities (e.g., urination), data from electronic medical/health records, demographic data, sensor data, and so forth.

The output of a digital twin that simulates an organic or living subject may take various forms corresponding to various physiological and/or symptomatic responses of the digital twin to the input parameters. In some embodiments, the number of outputs may correspond to the number of inputs, although this is not required. Examples of physiological responses that may be represented by output of the digital twin include, for instance, heart rate, temperature, respiration rate, insulin response, glucose levels, blood oxygen levels, pulmonary function (e.g., tidal volume), and so forth. In various implementations, output of a digital twin may include simulated electrocardiogram (“ECG”) signal(s), one or more simulated signals indicative of blood oxygen levels or saturation (e.g., SpO₂), etc. Symptomatic responses of a digital twin (which may be indicated and/or conveyed by output of digital twin) may include, for instance, restlessness, hunger, anxiety, thirst, drowsiness, itching, pain, etc. In some embodiments, the output of the digital twin may predict, or may be used to predict, progression of a condition such as a disease, deterioration of one or more components of a subject (e.g., internal organs), and so forth.

Generally, in one aspect, a method implemented using one or more processors may include: identifying one or more user needs of a user seeking to utilize a digital twin; identifying one or more situational needs of a subject simulated by the digital twin, wherein the one or more situational needs are identified based on data obtained from the subject; based on one or more of the user needs and one or more of the situational needs of the subject, selecting and applying one or more models of the digital twin to generate digital twin output, wherein the digital twin output simulates one or more aspects of the subject; and based on the digital twin output, providing visual or audible output to the user about the subject.

In various embodiments, the subject may include at least part of a patient, such as one or more internal organs. In various embodiments, the user may be the patient or a clinician that is treating the patient. In other embodiments, the subject may a machine or a vehicle, and the user may be an engineer, technician, or other entity interested in operation of the machine or vehicle.

In various embodiments, the method may include applying data indicative of one or more of the user needs and one or more of the situational needs of the subject as inputs across a machine learning model to generate model selection output, wherein the selecting is based on the model selection output. In various embodiments, the selecting may be based on a comparison of one or more of the user needs and one or more of the situational needs of the subject with one or more lookup tables.

In various embodiments, the method may further include refining the one or more situational needs of the subject based on the digital twin output. In various embodiments, the one or more user needs and one or more situational needs may be selected from an enumerated list of needs, and the one or more user needs are prioritized over the one or more situational needs.

In various embodiments, the method may further include: detecting a conflict between one or more of the user needs and one or more of the situational needs; and based on the detecting, refraining from providing visual or audible output to the subject about the subject, or prompt the user to reconsider one or more of the user needs. In various embodiments, the selecting may be further based on measures of quality associated with a plurality of models of the digital twin.

In addition, some implementations include one or more processors of one or more computing devices, where the one or more processors are operable to execute instructions stored in associated memory, and where the instructions are configured to cause performance of any of the aforementioned methods. Some implementations also include one or more non-transitory computer readable storage media storing computer instructions executable by one or more processors to perform any of the aforementioned methods.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

### BRIEF DESCRIPTION OF THE DRAWINGS

In the drawings, like reference characters generally refer to the same parts throughout the different views. Also, the drawings are not necessarily to scale, emphasis instead generally being placed upon illustrating various principles of the embodiments described herein.

FIG. 1 schematically illustrates an example environment in which selected aspects of the present disclosure may be implemented.

FIG. 2 schematically illustrates an example computing device implementing a user interface that is operable to facilitate selected aspects of the present disclosure.

FIGS. 3A and 3B depict example lookup tables that relate various aspects of the present disclosure to each other, in accordance with various embodiments.

FIG. 4 schematically depicts one example of how a subject may be classified into different states, each state being associated with a different level of monitoring of the subject.

FIG. 5 depicts an example method for practicing selected aspects of the present disclosure.

FIG. 6 depicts an example computing system architecture that may be employed to carry out selected aspects of the present disclosure.

### DETAILED DESCRIPTION

With increasing amounts of available data, mathematical models, and potential applications of those models, it is becoming more difficult to determine which functions and/or models of a digital twin to apply. In the healthcare context, for instance, it has become more difficult for clinicians to keep track of all the new model options. For patients who lack medical expertise, the increasing volume and/or variety of models may be even more confusing and/or intimidating. Accordingly, various embodiments and implementations of the present disclosure are directed to selecting and applying digital twin models in various contexts.

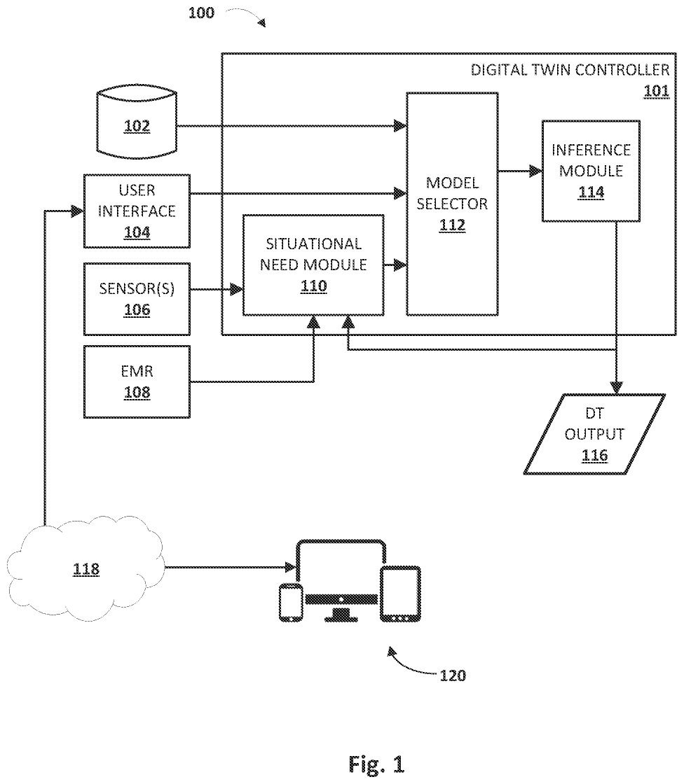

FIG. 1 depicts an example environment 100 in which selected aspects of the present disclosure may be implemented. The computing devices depicted in FIG. 1 (e.g., 101, 120) may include, for example, one or more of: a desktop computing device, a laptop computing device, a tablet computing device, a mobile phone computing device, a computing device of a vehicle of the user (e.g., an in-vehicle communications system, an in-vehicle entertainment system, an in-vehicle navigation system), a standalone interactive speaker (which in some cases may include a vision sensor), a smart appliance such as a smart television (or a standard television equipped with a networked dongle with automated assistant capabilities), and/or a wearable apparatus of the user that includes a computing device (e.g., a watch of the user having a computing device, glasses of the user having a computing device, a virtual or augmented reality computing device). Additional and/or alternative client computing devices may be provided.

A digital twin controller 101 may be implemented using one or more computing devices that form what is sometimes referred to as a “cloud” infrastructure, or simply the “cloud.” Digital twin controller 101 may include and/or have access to various sources of data, including a digital twin model index 102, a user interface module 104, one or more sensors 106, and/or one or more electronic medical records (“EMR”) 108 (which could also include electronic health records, or “EHRs”). Other data sources are also contemplated.

Digital twin model index (or “database”) 102 may include one or more trained models that may be employed as part of a digital twin. These models may be trained to generate output indicative of various predictions, prognoses, inferences, etc. The models in index 102 may take various forms, including but not limited to causal Bayesian networks, k-means models, decision trees (including random forests), support vector machines, convolutional neural networks (“CNNs”), and other types of neural networks such as feed-forward neural networks, recurrent neural networks, mechanistic models such as biophysical models, and so forth.

User interface module 104 may provide an interface to which one or more client devices 120 may connect over one or more local area and/or wide area networks 118. User interface module 104 may provide functionality that enables a user of a client device 120 to select user needs (described below) associated with use of a digital twin, to view output and/or results of techniques described herein, and so forth. For example, in some embodiments, user interface module 104 may serve up documents in hypertext markup language (“HTML”) or extensible markup language (“XML”) formats. These documents may convey outcomes and/or results of techniques described herein. In other implementations, user interface module 104 may provide other types of output, such as audio output, audio-visual output, haptic feedback, etc. User interface module 104 may in some embodiments provide a question-and-answer interface (visual or audio) that enables a subject (e.g., operating a client device 120) to answer a series of questions. These questions may seek information about the subject's needs (e.g., do they want to be notified of various things?), as well as about the subject's health in general.

Sensor(s) 106 may include any device or component that is configured to passively and/or actively collect measurements or other observations about a subject. In the healthcare context, one or more sensors 106 may provide various physiological information about a subject such as a patient. These sensors may include, but are not limited to, heart rate sensors (e.g., electrocardiograms, or “ECG”), blood oxygen sensors (e.g., pulse oximetry/SpO₂), glucose sensors, thermometers, sweat sensors, spirometry instrument(s), and so forth. These sensors 106 may be deployed in a healthcare environment, e.g., to take measurements during doctor's office or hospital visits. These sensors 106 may additionally or alternatively be deployed outside of a clinician environment, such as in a subject's home or residence. Some sensors 106 may be deployed as part of and/or in communication with mobile computing devices that may or may not be wearable, such as smart phones, smart watches, smart glasses, head-mounted displays, etc. Some sensors 106 may take the form of patches that are able to take measurements, and in some cases may also perform other functions such as administering medicine, facilitating monitoring of a vital sign (e.g., in conjunction with a connected computing device), and so forth.

EMRs 108 may be obtained from various sources, such as hospital information systems (“HIS”, not depicted), individual clinicians' offices, cloud-based patient data repositories, and so forth. An EMR 108 may include a variety of different information about a subject, such as height, weight, gender, reimbursement information, demographics, a clinician's composed notes about the subject, physiological measurements, medical history, lab results, prescriptions (past and present), treatment plans, data about adherence to treatment plans, diagnoses, family history, and so forth. In some embodiments, data from other sources like sensors 106 may be included in an EMR 108.

Digital twin controller 101 itself may include various modules that implement selected aspects of the present disclosure. For example, in FIG. 1, digital twin controller 101 includes a situational need module 110, a model selector 112, and an inference module 114. In some embodiments, index 102 and/or user interface module 104 may also be part of digital twin controller 101. In other embodiments, various aspects of digital twin controller 101 may be combined, omitted, or implemented elsewhere.

Situational need module 110 may be configured to determine, based on various inputs, situational or “contextual” needs of a subject at the moment. In some implementations, these situational needs may correspond to, coincide with, or otherwise be related to the user needs described previously. For example, in FIG. 1, situational needs module 110 receives, as input, data from sensor(s) 106, data from EMR(s) 108, and digital twin (“DT” in FIG. 1) output 116 generated by inference module 114. Based on these inputs, situational need module 110 may output one or more situational needs to model selector 112.

Model selector 112 may be configured to select, e.g., from model index 102, one or more digital twin models that should be implemented or applied based on various signals. In additional to situational needs provided by situational needs module 110, these signals may also include other signals described herein, such as user needs provided via user interface module 104. Model selector 112 may employ various techniques to select digital twin models to apply in various situations. These techniques may include but are not limited to heuristics, lookup tables, statistical models, causal graphs such as causal Bayesian networks and/or probabilistic graphical models, trained machine learning models (e.g., neural networks), decision trees, support vector machines, and so forth.

In some embodiments, the output of model selector 112 may include identifiers and/or data (e.g., weights) associated with one or more selected models from index 102. This output may be provided as input to inference module 114. Inference module 114 may then apply the selected model(s) and/or weights to various input data, such as from sources 102, 106, and 108, to generate DT output 116. This DT output 116 may predict, or may be used by one or more downstream components (not depicted) to predict, various behavioral and/or physical responses of the subject. For example, the DT output 116 may predict (or be used to predict) deterioration of one or more organ systems, to predict advancement of a chronic disease, to predict positive consequences of a subject's changing his or her behavior (eating healthy, quitting drinking or smoking), and so forth.

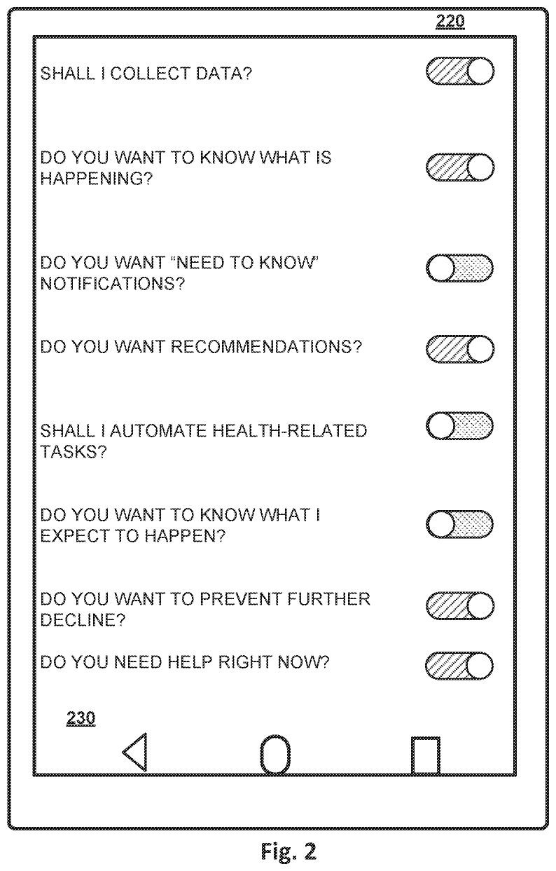

FIG. 2 depicts an example client device 220 in the form of a smart phone or tablet computer. Client device 220 includes a touchscreen 230 that is currently rendering a graphical user interface (“GUI”). The GUI is operable by a user such as a subject (or a clinician in some cases) to make decisions about what kind of information the user would like to see, and what kind of information the user would not like to see. Put another way, these questions solicit user needs, whether the user is the subject or someone else (e.g., family member, clinician). These questions are merely for illustrative purposes, and are not meant to be limiting.

In this example, the first question is, “Shall I collect data?” This question may seek to address, for instance, privacy concerns, and whether the user is comfortable with data being shared as part of an aggregated data set associated with, for instance, a population of subjects (this question might not be applicable where, for instance, the subject is a machine or vehicle). For example, a subject may or may not be comfortable with sensor data that is obtained from sensor(s) 106 being collected for use by a third party entity that manages digital twin controller 101. In this example, the user has toggled a corresponding toggle switch to the right, indicating the user acquiesces to such data collection. In some embodiments, this may result in the collected data being shared (e.g., anonymously) across a population of subjects, e.g., for research purposes, for purposes of training machine learning model(s), etc.

The next question is, “Do you want to know what is happening?” This is another way of asking the subject whether they wish to receive information about what is happening in their body, e.g., in response to their lifestyle choices. This user has elected to receive this information.

The next question is, “Do you want to receive ‘need to know’ notifications?” For example, does the user wish to receive warnings if one or more digital twin models generates output that triggers a health warning such as a diagnosis of some condition? Here, the user has elected not to receive this information. They may not react well emotionally to such notifications, or they may be comfortable with their own health status and do not wish to be bothered with such notifications. Or, they may even not place much confidence in such notifications or the digital twin models that underlie them.

The next question is, “Do you want recommendations?” This question may seek to learn whether the user wants advice, guidance, motivation, etc., in order to reach various health-related goals. These recommendations/guidance may be identified and/or selected based on digital twin model output. In this example the user has selected yes.

The next question is, “Shall I automate health-related tasks?” In other words, would the user like to have various health-related tasks automated in response to output of digital twin model(s)? These health-related tasks may include, for instance, automatically scheduling a doctor's appointment, making an appointment with a smoking cessation provider, automatically requesting a prescription from a doctor, sending an automated message to another person (e.g., family member, caregiver), etc. (many of these tasks may alternatively be provided as recommendations to the user as described in the previous paragraph). Some users may desire that some or all of these tasks be handled automatically, and others may not. In this example the user has elected not to have health-related tasks automated.

The next question is, “Do you want to know what I expect to happen?” In other words, does the user want predictions to be made and/or conveyed to them about what various digital twin models predict about the future of the subject's health? Once again the user has selected not to receive this information. The next question is, “Do you want to prevent further decline?” The user's response to this question may determine whether the user will receive information and/or be referred to pertinent clinician(s) to prevent further decline of, for instance, an internal organ system. The final question is, “Do you need help right now?” If a user toggles this on, in some embodiments, the user may be connected with a first responder or other personnel that can assist the user right away.

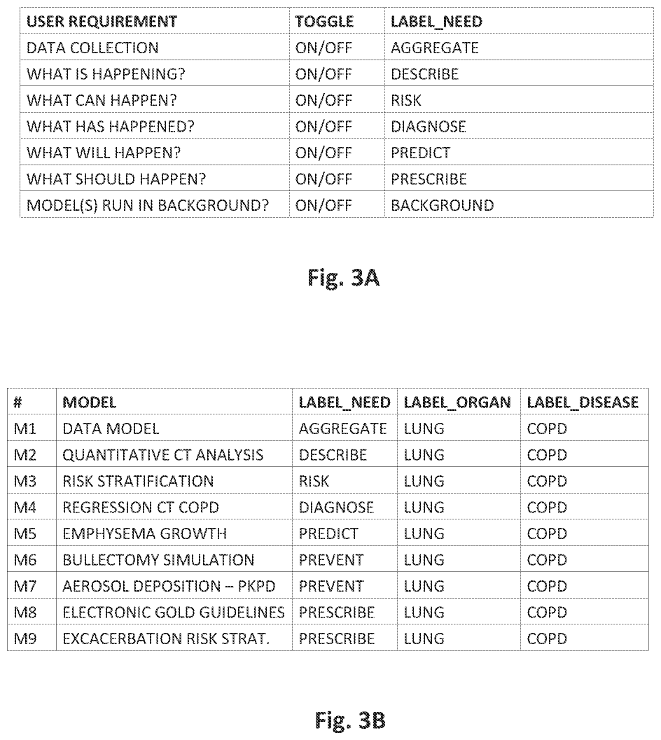

FIGS. 3A and 3B each includes a lookup table that, together, demonstrate one example of how user needs and situational needs may be related and/or examined relative to each other. These tables also provide non-limiting examples of digital twin models that may be deployed as part of digital twins. In FIG. 3A, the lookup table relates to user needs, e.g., provided at user interface module 104. The lookup table in FIG. 3A includes, under the “User Requirement” column, questions that may or may not correspond in whole or in part to questions such as those depicted in FIG. 2. The “Toggle” column indicates user selection or de-selection of a particular option. The “LABEL_NEED” column indicates which user need each row corresponds to. In this lookup table and elsewhere herein, labels may be manually curated and/or automatically extracted from medical documents, e.g., using techniques such as natural language processing.

The table in FIG. 3A includes a “data collection” user requirement that corresponds to the first question in FIG. 2, “Shall I collect data?” This user requirement is associated with a user need of “aggregate.” An aggregate user need may correspond to a choice of whether data (e.g., from sensors and/or EMR/EHRs) collected for purposes of operating digital twin(s) and/or their constituent models should be made available (e.g., anonymously) as part of aggregated data associated with a population of subjects. Thus, the user can toggle on/off whether this data is to be shared (e.g., anonymously) as part of a larger data set. The other user needs in FIG. 3A operate similarly to allow the user to choose when data such as digital twin model output is generated and/or output to the user, and when it is not.

The lookup table in FIG. 3B relates situational needs with digital twin models, and can be used to cross-reference situational needs with user needs in some embodiments. The table in FIG. 3B includes, for each row, a digital twin model and its associated situational need. For example, the M1 “Data Model” is a general purpose model that is associated with an “aggregate” situational need. As alluded to previously, an “aggregate” situational need may indicate a general need for information from the subject to be used (e.g., anonymously) as part of a larger population. In some embodiments there is always an aggregate situational need because it is almost always beneficial to share subject data whenever possible. In other embodiments, a measure of aggregate situational need may depend on, for instance, the availability of data in various domains.

As another example, the M4 model, “Regression CT COPD,” may be a regression-based machine learning model (e.g., logistical regression) that takes as inputs various CT biomarkers from (e.g., lung cancer screening Computed Tomography scans etc.), and generates output that predicts and/or can be used to predict a diagnosis of the subject. This model may be associated with a situational need of “diagnose.” The two right columns of FIG. 3B indicate which organ and condition, respectively, the models target. In other implementations, other models that target other conditions and/or organs may be included in such a table.

In various embodiments, user needs and situational needs may be compared to determine which digital twin models are applied, which are not applied, and/or which are applied and output only to clinicians (rather than to the subjects). In some such embodiments, user needs may override situational needs. For example, situational needs may indicate a possibility that a subject has a condition, and diagnosis models may normally be applied in such a situation. However, suppose the subject has opted out of receiving automated diagnoses (e.g., “What has happened” in FIG. 3A is toggled off). In such a scenario, the user's stated need may override the situational need (although in some cases a clinician may be informed of the situation). In other such embodiments, situational needs may override user needs. For example, if a user elected not to automate health-related tasks but output of a digital twin model indicates a medical emergency with a high level of confidence, first responders may be called regardless of the subject's stated preference.

As one non-limiting example, suppose a female patient has a respiratory disease. EMR 108 data, which could in some instances include reimbursement data associated with this patient, may indicate that in the last few years the patient increasingly needs bronchodilators. This data may be labeled “lung” in some embodiments. Based on these new data, the situational need of the patient may be changed, e.g., by situational need module 110, from “describe” to “risk.” If the patient has toggled the user need “risk” to on (“What can happen?” in FIG. 3A), a risk stratification model (e.g., M3 in FIG. 3B) for lungs may be selected by model selector 112 and applied by inference module 114.

Suppose output of this model M3 indicates that the patient might have chronic obstructive pulmonary disease (“COPD”). Since this COPD risk is new information, situational need module 112 may change the situational need label from “risk” to “diagnose.” If the user requirement label “diagnose” is “on” (“What has happened?” in FIG. 3A), a regression model of the type diagnostic (M4) may be selected, e.g., by model selector 112. Suppose output of this M4 model confirms that the user has COPD with a high probability. In the same manner, model selector 112 may continue iterating through predictive, preventive and prescriptive models, assuming the user has not opted out of any of these. For example, the M5 model may be selected by model selector 112 and applied by inference module 114 to predict the progression of COPD. The M6 and/or M7 model may be selected by model selector 112 and applied by inference module 114 to determine what interventions are needed. The M8 and/or M9 model may be selected by model selector 112 and applied by inference module 114 to prescribe when action needs to be taken to prevent a hospital admission for a lung exacerbation. And in many cases, the user's personal preferences that are indicated by their selections of user needs may override these situational needs.

In some implementations, at every step, information may or may not be provided to the user as output via user interface module 104, depending on the user needs. In some embodiments, at every step the user may be asked to maintain or adapt his or her user needs. For example, suppose COPD is detected in the patient, and that the patient has previously opted out of receiving information from models labeled for prediction. The user may be asked whether he or she wants to know what will happen (predict) and/or what should happen in order to prevent a further decline of lung function. For a more experienced or expert user (e.g., a clinician), a list of user/situational needs can be presented directly.

The lookup tables in FIGS. 3A and 3B are provided for illustrative purposes only and are not meant to be limiting. In various embodiments, the “pool” or “library” of digital twin models that are available for use at any given time can be dynamic, and can change based on a variety of factors. Suppose a new sensor is activate to obtain additional data from a subject. For example, suppose a patient purchases a new smart watch with a type of sensor that the patient didn't have previously. In such a circumstance, one or more additional digital twin models may become available and/or applicable. For example, the purchase of the smart watch (and its constituent sensor) may be accompanied with a subscription to one or more new digital twin models. Or, the newly-available sensor data may unlock one or more existing digital twin models that were previously locked. Situational needs also change, and as they change, various digital twin models may be unlocked or locked as the case may be. For example, if a patient is detected to be developing a condition over time, digital twin models associated with treatment and/or monitoring of that condition may become unlocked and available to the patient, e.g., automatically, in response to the patient's consent, in response to a purchase, etc.

Additionally, in some embodiments, user needs, and/or situational needs (or even digital twin models) may be weighted relative to each other. Rather than user needs simply overriding situational needs, in some embodiments, user need and situational need weights may be compared to resolve a potential conflict. If a difference or delta between the situational and user need weights exceeds some threshold, the situational needs may override the user needs in spite of the user needs being in apparent conflict with the situational needs. Alternatively, if the delta between the situational and user need weights exceeds some threshold, the user may be prompted to reconsider their user needs.

In some embodiments, a library of one or more digital twin models may be stored locally on a client device (120 in FIG. 1). As the circumstances of the user of the client device change, that local library of digital twin models may be updated, e.g., with new models being downloaded, existing models being replaced or deleted, existing models being updated, etc. In some embodiments, those changing circumstances may include the situational needs described herein. As one example, suppose a subject develops prediabetes. A library of digital twin models stored on the patient's smart phone—or available across a coordinated ecosystem of client devices controlled by the patient—may be updated to include one or more digital twin model(s) that are pertinent to monitoring and/or treatment of high blood sugar. As another example, suppose a subject moves to a different region where particular allergens are more prevalent. A library of digital twin models stored on (or available to) the subject's computing device(s) may be updated to include digital twin model(s) that are pertinent to monitoring and/or treatment of allergies and/or asthma.

In additional to or instead of using lookup tables as shown in FIGS. 3A-B, in some embodiments, model selector 112 may employ machine learning model(s) that are trained to generate output indicative of which models should be used in a given situation. In some embodiments, for training, validation, and testing of these machine learning model(s), training data inputs may include any combination of data from sensor(s) 106, user needs (e.g., provided at user interface 104), situational needs 110, EMR data 110, and/or digital twin output 116. This training data may be annotated, for instance, with “ground truth” digital twin model identifiers (e.g., “M1,” “M2,” etc.) that identify the machine learning model(s) that should be applied in each instance. The output of these machine learning model(s) may be predicted digital twin model identifiers. The predicted digital twin model identifiers may be compared to the ground truth digital twin model identifiers. Based on the error or loss that results from the comparison, the machine learning model(s) may be trained, e.g., using techniques such as gradient descent, back propagation, etc. Suitable machine learning algorithms may include but are not limited to k-means, Gaussian mixture models (“GMM”) (e.g., with expectation maximization), decision trees (including random forest), support vector machines, convolutional neural networks, and other neural networks.

The following is another example that illustrates how techniques described herein may be employed to automatically select which digital twin model(s) are applied, which are not, and/or which are applied but the output is only provided to a clinician, not to the subject (e.g., patient) modeled by the digital twin. For this example, suppose a female patient has a smoking history of thirty pack-years and decides to participate in a lung cancer screening program after seeing a recruitment video on local television. As a reward for her participation, the patient receives free digital twin software (e.g., a personal health app) which she installs on her mobile device.

As part of the lung cancer screening program, annual low dose computed tomography (“CT”) scans are taken from the patient. A screening radiologist interprets the CT scan and writes a report and a letter to the general practitioner. These documents or portions thereof may be stored, for instance, as EMR(s) 108 of the patient. Suppose further that the patient agrees, e.g., using a GUI such as that depicted in FIG. 2 provided by user interface module 104, that her data should be made available for aggregation and analysis by data mining software (e.g., anonymously). For example, she may toggle “yes” for the first question in FIG. 2 (e.g., an “aggregate” user need). As a result, the lung cancer screening population is able to benefit, e.g., by way of learning by similarity network models (e.g., K-means, GMM, etc.). The patient also anticipates that, perhaps in a couple of years, she might benefit herself when her data is compared to similar patients.

The patient also likes the idea of having a digital twin to watch over her lung health. Accordingly, she approves to apply quantitative analysis of her CT scans, e.g., by computational models (“QCT”), e.g., by toggling “yes” the second question (“Do you want to know what is happening”) in FIG. 2 (e.g., a “describe” user need). Besides suspicious lung nodules, QCT software can analyze the lung anatomy in general, and detect signs of pathology, i.e. lung diseases such as emphysema. Additionally, QCT based machine learning models may be trained to generate output indicative of lung function without requiring the patient to undergo spirometry. Because CT scans are made yearly, longitudinal information is available that reflects the development of the patient's lung health and function.

Besides early signs of lung disease, QCT combined with regression models can also be used to diagnose, for instance, pulmonary conditions such as COPD. Suppose the patient decides that other than the development of her lung health in general (e.g., user need of “describe”), she does not want to be pushed more information about her lungs. In particular, the patient does not wish to be proactively informed of diagnoses predicted by digital twin model(s) (e.g., user need of “diagnose” in FIG. 3A), at least as long as she does not have or suffer from symptoms. This may be important to her for purposes of alleviating anxiety, and/or because it might affect her employability and/or insurability, as well as her ability to negotiate for a housing mortgage. Accordingly, she may select “no” for the third question in FIG. 2.

However, the patient may rely on her digital twin to provide recommendations, such as “quit smoking” (as opposed to diagnoses such as “you have COPD”). Accordingly, she may select “yes” for the fourth question in FIG. 2. Based on her demographic and lifestyle data, and the application of a risk model, her digital twin may assess an increased risk for the development of pulmonary disease such as COPD and may recommend that the patient participate in a “quit smoking” program.

Suppose the patient does not want health-related tasks such as scheduling a doctor's appointment or an appointment with a smoking cessation provider to be automated. Perhaps she'd rather consult her general practitioner and follow their recommendation for a suitable program than follow a computer's recommendation. Accordingly, she may select “no” to the fifth question in FIG. 2.

Similar to her need and underlying motivations for not wanting to get notifications when her digital twin diagnoses a disease, the patient may not want to know future predictions. For example, it is possible to predict/give a prognosis of accelerated lung function decline based on low dose CT scans in lung cancer screening programs. Accordingly, the patient may select “no” to the sixth question in FIG. 2 (e.g., user need of “predict” in FIG. 3A).

Suppose that after passage of a number of years, the patient develops symptoms and a low-grade COPD is diagnosed by her pulmonologist. The patient may desire to prevent the development of more severe COPD. Accordingly, she may take various remedial actions, such as quitting smoking. She also may update her user needs to allow her digital twin to optimize the therapy to treat her COPD, e.g., by selecting “yes” to the seventh question in FIG. 2 (e.g., user need of “prescribe” in FIG. 3A). As a result of her opting in, a digital twin model of the airflow in her lungs may be used, e.g., in combination with a pharmacokinetic model, to model the deposition of aerosol drug particles in her lungs, and translocation in her body. Her yearly CT scans are also available, and include information on the position and phenotypes of the diseased regions in her lung lungs. With this data and the digital twin airflow and pharmacokinetic models, the medication type, dose, and/or delivery mechanism may be optimized to prevent the development of more severe COPD.

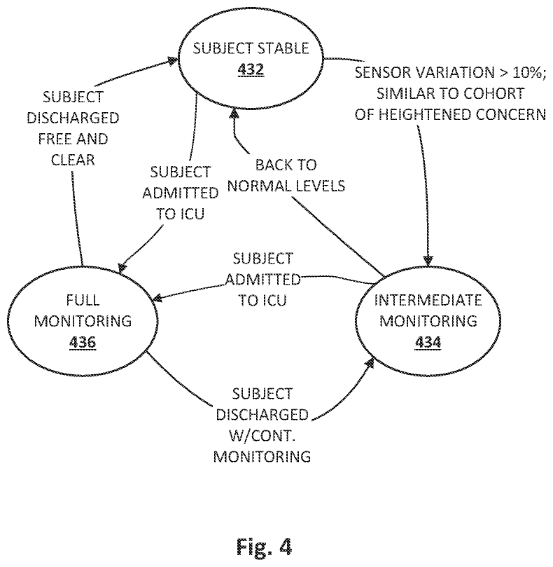

FIG. 4 depicts another example of how digital twin models may be selected by model selector 112 and applied by inference module 114, in accordance with various embodiments. In FIG. 4, it can be assumed that the subject has opted in to everything, e.g., by selecting “yes” to every question in FIG. 2. Because there are no user needs that may override and/or cancel a situational need, situational needs may be controlling. However, constant monitoring and digital twin application can still burden computing resources, especially to the extent these operations are performed on client devices 120 that may be battery-powered and/or may be otherwise resource-constrained. Accordingly, FIG. 4 demonstrates one example of how digital twin models may be selected and applied as a subject's situation changes (as reflected in changing situational needs).

In this example, a subject who is similar to a cohort of other subjects that are not in need of intensive monitoring and/or treatment (e g., a healthy cohort of patients) may be placed in the subject stable state 432. Additionally or alternatively, so long as data from one or more of the subject's sensors 106 has less than some threshold variance (e.g., less than 10%) over some period of time (e.g., a week), the subject may be placed in the subject stable state 432. In the subject stable state 432, general status monitoring may be performed (e.g., data collection) to provide descriptive information (e.g., “describe” need in FIGS. 3A-B). However, digital twin models that seek to infer information about specific ailments may not be applied, e.g., to conserve computing resources, to preserve the subject's privacy, to avoid intrusion on the subject, etc. Rather, in some embodiments, general medical guidelines may be applied instead of predictive digital twin models.

When the subject shows unexpected behavior or symptoms, the subject may be transitioned from the subject stable state 432 to the intermediate monitoring state 434. In other words, the level of intelligence applied in association with the subject's digital twin may be scaled up. This may occur, for instance, when the subject's sensor data varies more than some threshold in some period of time (e.g., more than 10% variance in a week). This may alternatively occur when, for instance, the subject becomes more similar to a cohort of subjects in need of heightened treatment/monitoring than a cohort of healthy subjects. Similarity of the patient to various cohorts may be determined using various techniques, such as K-means clustering, GMM, cosine similarity, Euclidian distance in latent space, etc. For example, the subject's data may be used to generate an embedding. A distance between this embedding and clusters of embeddings that correspond to different cohorts of subjects may be calculated to identify the cluster (and cohort) to which the subject is most similar.

In the intermediate monitoring state 434, various predictive digital twin models may now be applied in order to gain additional information that may explain changes in the subject's behavior, symptoms, and/or well-being in general. For example, one or more biophysical digital twin models may be applied to interpret physiological data captured by a smart watch worn by the patient. In some cases this interpretation may include diagnosing the subject with condition(s), predicting exacerbation of a condition, etc. If the subject's data returns to lower levels of variance, and/or the subject's data indicates they have returned to being most similar to a healthy cohort, as shown in FIG. 4, the subject may be transitioned back to subject stable state 432.

However, suppose the subject is admitted to an intensive care unit (“ICU”) of a hospital, or is the subject of an emergency call to first responders. Under such circumstance, the subject may be transitioned—from the intermediate monitoring state 434 or the subject stable state 432 as the case may be—to a full monitoring state 436. In the full monitoring state, the full capabilities/intelligence of the subject's digital twin may be activated in order to determine what needs to be done to maximize the likelihood of a positive outcome for the subject. For example, an intervention may be prescribed based on successful interventions in similar scenarios (which similar to above may be determined using clustering and/or similarity measures in latent space). If the subject is ultimately discharged free and clear, they may be transitioned back to the subject stable state 432. Perhaps more likely, if the subject is discharged with clinician instructions for continued monitoring and/or rehabilitation, the subject may be transitioned back to the intermediate monitoring state 434.

In some embodiments, digital twin models may be selected by model selector 112 in accordance with various heuristics that select models based on availability of models or the data they operate on, applicability of the models, and/or quality of the data and/or the digital twin models themselves. In some embodiments this selection may be made on a hierarchal basis.

For example, in some embodiments, a subject that indicates no specific health concerns may be considered for analysis using a relatively “general purpose” candidate digital twin model, such as a model that predicts general cardiac risk scores, as opposed to a model that predicts the specific risk of a stroke. But the candidate digital twin model may be scrutinized for its applicability and/or quality first. If the most influential data points for that model are not available (e.g., because the subject doesn't wear the applicable sensors or doesn't follow a clinician's orders to self-report), the candidate digital twin model may not be effective and consequently may not be selected by model selector 112. For example, when no lab values for cholesterol are available, digital twin models for which cholesterol is the most sensitive parameter may not be selected by model selector 112.

Additionally or alternatively, the data that is available for the subject, such as the subject's age, gender, demographics, etc., may be compared with that associated with the candidate digital twin model to ensure the candidate digital twin model will be effective. Put another way, was the digital twin model generated/validated for a population or cohort in which the subject would be a good fit? As an example, different risk prediction models may exist for women than for men.

In some implementations, the availability/quality of the data and/or the digital twin model(s) may be used, e.g., by model selector 112, as a preselection to narrow down the candidates. In some such embodiments, other criteria may then be applied to select which of the remaining candidate digital twin models should be selected by model selector 112 and applied by inference module 114. These other criteria, may include, for instance, indicators of the models' performance, such as accuracy measures, false positive rates, an F-1 score, an area under a receiver operating characteristic (“ROC”) curve, etc. More generally, other reputational signals that may be considered include but are not limited to digital twin model reputation among clinicians or government agencies, government clearance (e.g., by the United States Food and Drug Administration, or “FDA”), costs, clinical evidence, etc. Alternatively, a subject may be presented with multiple candidate digital twin model options, and may select from among them based on their accuracies, associated fees, privacy protections, etc.

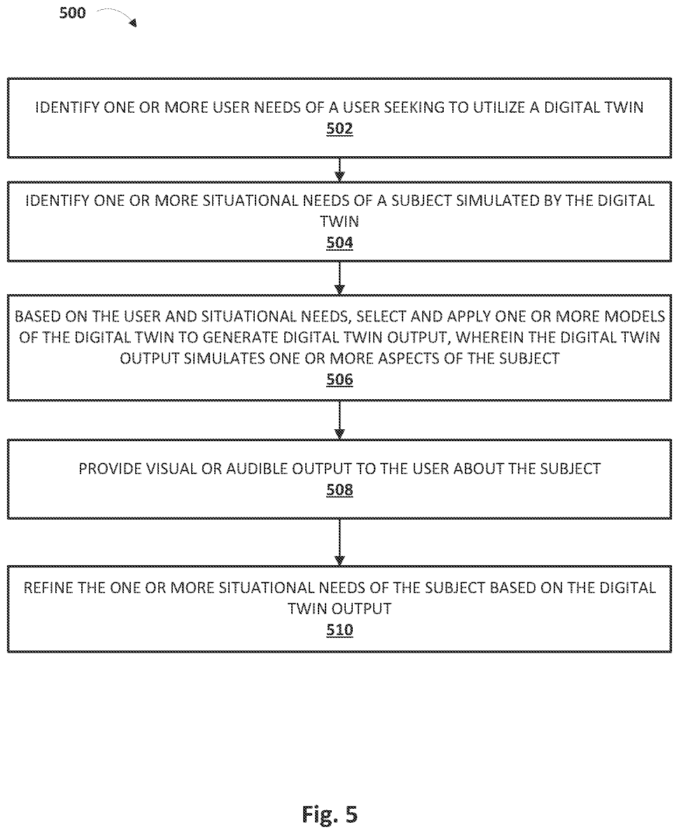

FIG. 5 illustrates a flowchart of an example method 500 for practicing selected aspects of the present disclosure to train a pain estimation machine learning model. The steps of FIG. 5 can be performed by one or more processors, such as one or more processors of the various computing devices/systems described herein. For convenience, operations of method 500 will be described as being performed by a system configured with selected aspects of the present disclosure, such as digital twin controller 101. Other implementations may include additional steps than those illustrated in FIG. 5, may perform step(s) of FIG. 5 in a different order and/or in parallel, and/or may omit one or more of the steps of FIG. 5.

At block 502, the system may identify one or more user needs of a user seeking to utilize a digital twin, e.g., from input data received at user interface 104. For example, a user such as the subject modeled by a digital twin and/or a another person (e.g., a clinician that treats the subject) may operate a GUI such as that depicted in FIG. 2 to select what needs/wants the user has regarding their digital twin model usage. As noted herein, in many contexts the subject may be a patient (human or otherwise), but can also be an inorganic system such as a vehicle. When the subject is a vehicle or other inorganic system, the user may be a person such as a technician or engineer that is interested in simulating aspects of the system. In such case, the user needs may be those of the technician/engineer.

At block 504, the system may identify one or more situational needs of a subject simulated by the digital twin. In various embodiments, the one or more situational needs may be identified, e.g., by situational need module 110, based on data obtained from the subject from sources such as sensor(s) 106 and/or EMR(s) 108, from data provided by the subject via user interface 104 (e.g., answers to questionnaire), and/or based on feedback from digital twin model output 116.

Based on one or more of the user needs and one or more of the situational needs of the subject, at block 506, the system may, by way of model selector 112 and inference module 114, respectively, select and apply one or more models of the digital twin to generate digital twin output. In various embodiments, the digital twin output may simulate one or more aspects of the subject, such as their behavior, symptoms, vital signs, etc. In some embodiments, the selecting of block 506 may include the system applying data indicative of one or more of the user needs and one or more of the situational needs of the subject as inputs across a machine learning model to generate model selection output. This model(s) that are selected and applied may be based on the model selection output. Additionally or alternatively, the selecting of block 506 may be based on a comparison of one or more of the user needs and one or more of the situational needs of the subject with one or more lookup tables, such as those depicted in FIGS. 3A-B.

Based on the digital twin output, at block 508, the system, e.g., by way of user interface 104, may provide visual or audible output to the user about the subject. In some embodiments, at block 510, the system may refine the one or more situational needs of the subject based on the digital twin output. Other operations of method 500 may then be performed again to select and apply different digital twin models.

In some embodiments, the system may detect a conflict between one or more of the user needs and one or more of the situational needs. For example, the situational needs may suggest applying a diagnostic digital twin model to data to determine whether the subject has a condition. However, the subject may have indicated they do not wish to be informed of such developments. In some embodiments, the user need may override the situational need, and the system may refrain from providing output to the subject. However, in some such embodiments, the system may still inform another party, such as the subject's clinician, so that the clinician has the option of intervening even where the subject chooses to remain uninformed,

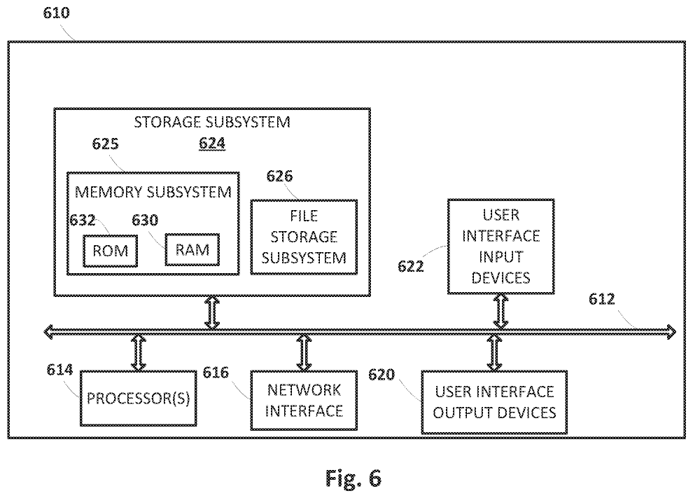

FIG. 6 is a block diagram of an example computing device 610 that may optionally be utilized to perform one or more aspects of techniques described herein. Computing device 610 typically includes at least one processor 614 which communicates with a number of peripheral devices via bus subsystem 612. These peripheral devices may include a storage subsystem 624, including, for example, a memory subsystem 625 and a file storage subsystem 626, user interface output devices 620, user interface input devices 622, and a network interface subsystem 616. The input and output devices allow user interaction with computing device 610. Network interface subsystem 616 provides an interface to outside networks and is coupled to corresponding interface devices in other computing devices.

User interface input devices 622 may include a keyboard, pointing devices such as a mouse, trackball, touchpad, or graphics tablet, a scanner, a touchscreen incorporated into the display, audio input devices such as voice recognition systems, microphones, and/or other types of input devices. In general, use of the term “input device” is intended to include all possible types of devices and ways to input information into computing device 610 or onto a communication network.

User interface output devices 620 may include a display subsystem, a printer, a fax machine, or non-visual displays such as audio output devices. The display subsystem may include a cathode ray tube (CRT), a flat-panel device such as a liquid crystal display (LCD), a projection device, or some other mechanism for creating a visible image. The display subsystem may also provide non-visual display such as via audio output devices. In general, use of the term “output device” is intended to include all possible types of devices and ways to output information from computing device 610 to the user or to another machine or computing device.

Storage subsystem 624 stores programming and data constructs that provide the functionality of some or all of the modules described herein. For example, the storage subsystem 624 may include the logic to perform selected aspects of the method of FIG. 5, as well as to implement various components depicted in FIGS. 1-4.

These software modules are generally executed by processor 614 alone or in combination with other processors. Memory 625 used in the storage subsystem 624 can include a number of memories including a main random access memory (RAM) 630 for storage of instructions and data during program execution and a read only memory (ROM) 632 in which fixed instructions are stored. A file storage subsystem 626 can provide persistent storage for program and data files, and may include a hard disk drive, a floppy disk drive along with associated removable media, a CD-ROM drive, an optical drive, or removable media cartridges. The modules implementing the functionality of certain implementations may be stored by file storage subsystem 626 in the storage subsystem 624, or in other machines accessible by the processor(s) 614.

Bus subsystem 612 provides a mechanism for letting the various components and subsystems of computing device 610 communicate with each other as intended. Although bus subsystem 612 is shown schematically as a single bus, alternative implementations of the bus subsystem may use multiple busses.

Computing device 610 can be of varying types including a workstation, server, computing cluster, blade server, server farm, or any other data processing system or computing device. Due to the ever-changing nature of computers and networks, the description of computing device 610 depicted in FIG. 6 is intended only as a specific example for purposes of illustrating some implementations. Many other configurations of computing device 610 are possible having more or fewer components than the computing device depicted in FIGS. 6.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to.

Only the transitional phrases “consisting of” and “consisting essentially of” shall be closed or semi-closed transitional phrases, respectively, as set forth in the United States Patent Office Manual of Patent Examining Procedures, Section 2111.03. It should be understood that certain expressions and reference signs used in the claims pursuant to Rule 6.2(b) of the Patent Cooperation Treaty (“PCT”) do not limit the scope.

What is claimed is:

1. A method implemented using one or more processors, the method comprising:
identifying one or more user needs of a user seeking to utilize a digital twin;
identifying one or more situational needs of a subject simulated by the digital twin, wherein the one or more situational needs are identified based on data obtained from the subject;
based on one or more of the user needs and one or more of the situational needs of the subject, selecting and applying one or more models of the digital twin to generate digital twin output, wherein the digital twin output simulates one or more aspects of the subject; and
based on the digital twin output, providing visual or audible output to the user about the subject.

2. The method of claim 1, wherein the subject comprises at least part of a patient.

3. The method of claim 2, wherein the user comprises the patient or a clinician that is treating the patient.

4. The method of claim 1, wherein the subject comprises a machine or a vehicle.

5. The method of claim 1, further comprising applying data indicative of one or more of the user needs and one or more of the situational needs of the subject as inputs across a machine learning model to generate model selection output, wherein the selecting is based on the model selection output.

6. The method of claim 1, wherein the selecting is based on a comparison of one or more of the user needs and one or more of the situational needs of the subject with one or more lookup tables.

7. The method of claim 1, further comprising refining the one or more situational needs of the subject based on the digital twin output.

8. The method of claim 1, wherein the one or more user needs and one or more situational needs are selected from an enumerated list of needs, and the one or more user needs are prioritized over the one or more situational needs.

9. The method of claim 1, further comprising:
detecting a conflict between one or more of the user needs and one or more of the situational needs; and
based on the detecting, refraining from providing visual or audible output to the subject about the subject, or prompt the user to reconsider one or more of the user needs.

10. The method of claim 1, wherein the selecting is further based on measures of quality associated with a plurality of models of the digital twin.

11. A system comprising one or more processors and memory storing instructions that, in response to execution of the instructions by the one or more processors, cause the one or more processors to:
identify one or more user needs of a user seeking to utilize a digital twin;
identify one or more situational needs of a subject simulated by the digital twin, wherein the one or more situational needs are identified based on data obtained from the subject;
based on one or more of the user needs and one or more of the situational needs of the subject, select and apply one or more models of the digital twin to generate digital twin output, wherein the digital twin output simulates one or more aspects of the subject; and
based on the digital twin output, provide visual or audible output to the user about the subject.

12. The system of claim 11, wherein the subject comprises a patient.

13. The system of claim 12, wherein the user comprises the patient or a clinician that is treating the patient.

14. At least one non-transitory computer-readable medium comprising instructions that, in response to execution of the instructions by one or more processors, cause the one or more processors to:
identify one or more user needs of a user seeking to utilize a digital twin;
identify one or more situational needs of a subject simulated by the digital twin, wherein the one or more situational needs are identified based on data obtained from the subject;
based on one or more of the user needs and one or more of the situational needs of the subject, select and apply one or more models of the digital twin to generate digital twin output, wherein the digital twin output simulates one or more aspects of the subject; and
based on the digital twin output, provide visual or audible output to the user about the subject.

15. The at least one non-transitory computer-readable medium of claim 14, further comprising instructions to apply data indicative of one or more of the user needs and one or more of the situational needs of the subject as inputs across a machine learning model to generate model selection output, wherein the selecting is based on the model selection output.
